(19) United States
(12) Patent Application Publication
(10) Pub. No.: US 2019/0192861 A1
(43) Pub. Date: Jun. 27, 2019

(54) CONNECTOR ASSEMBLIES WITH NOVEL SEAL FOR ELECTRICAL STIMULATION SYSTEMS AND METHODS OF MAKING AND USING SAME

(71) Applicant: Boston Scientific Neuromodulation Corporation, Valencia, CA (US)

(72) Inventors: Thomas Paul Lopez, Sunland, CA (US); Jeffery Van Funderburk, Stevenson Ranch, CA (US)

(21) Appl. No.: 16/223,745

(22) Filed: Dec. 18, 2018

Related U.S. Application Data
(60) Provisional application No. 62/609,915, filed on Dec. 22, 2017.

(51) Int. Cl. A61N 1/375; A61N 1/36

(52) U.S. Cl. CPC A61N 1/3752; A61N 1/0553; A61N 1/36128

(57) ABSTRACT

A connector assembly includes an elongated connector housing and defines a port at one end of the connector housing for receiving a proximal end of a lead or lead extension; a lumen that extends from the port along at least a portion of the length of the connector housing; connector contacts axially spaced-apart and disposed along the lumen for coupling to a proximal end of a lead or lead extension; and non-conductive spacers disposed between adjacent connector contacts. Each of the spacers includes an outer tubular extension, an inner tubular extension, and a connection region coupling the outer tubular extension to the inner tubular extension. The inner tubular extension has a free end and the inner tubular extension is configured and arranged to form a seal against the proximal end of the lead or lead extension when inserted into the lumen.

### CROSS-REFERENCE TO RELATED APPLICATIONS

This application claims the benefit under 35 U.S.C. § 119(e) of U.S. Provisional Patent Application Ser. No. 62/609,915, filed Dec. 22, 2017, which is incorporated herein by reference.

### FIELD

The present invention is directed to the area of implantable electrical stimulation systems and methods of making and using the systems. The present invention is also directed to connectors utilizing a novel spacer design, as well as methods of making and using the same.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Stimulation of the brain, such as deep brain stimulation, can be used to treat a variety of diseases or disorders.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a connector assembly including an elongated connector housing having a first end, a second end, and a length, the connector housing defining a port at the second end of the connector housing, the port configured for receiving a proximal end of a lead or lead extension; a lumen that extends from the port along at least a portion of the length of the connector housing; connector contacts axially spaced-apart and disposed along the lumen such that the connector contacts are each exposed to the lumen, the connector contacts configured for coupling to a proximal end of a lead or lead extension when the proximal end of the lead or lead extension is inserted into the lumen; and non-conductive spacers disposed between adjacent connector contacts. Each of the spacers includes an outer tubular extension, an inner tubular extension, and a connection region coupling the outer tubular extension to the inner tubular extension. The inner tubular extension has a free end and the inner tubular extension is configured and arranged to form a seal against the proximal end of the lead or lead extension when inserted into the lumen.

In at least some embodiments, the outer tubular extension is configured and arranged to form a seal against the connector housing. In at least some embodiments, the inner tubular extension forms a ring. In at least some embodiments, the outer tubular extension forms a ring. In at least some embodiments, the inner tubular extension and the outer tubular extension have an equal thickness when a lead or lead extension is not inserted into the lumen. In at least some embodiments, the outer tubular extension has a free end. In at least some embodiments, the inner tubular extension or the outer tubular extension or both the inner and outer tubular extensions have two free ends and the connection region couples an intermediate portion of the outer tubular extension to an intermediate portion of the inner tubular extension.

In at least some embodiments, the inner tubular extension is configured and arranged to stretch when the proximal end of the lead or lead extension is inserted into the lumen. In at least some embodiments, the connector assembly is configured and arranged so that a one of the connector contacts acts as a stop to stretching of the inner tubular extension of a one of the spacers as the proximal end of the lead or lead extension is inserted into the lumen.

In at least some embodiments, the connector assembly is configured and arranged so that a one of the connector contacts acts as a stop to retraction of the inner tubular extension of a one of the spacers as the proximal end of the lead or lead extension is removed into the lumen. In at least some embodiments, the outer tubular extension extends further in an axial direction than the inner tubular extension. In at least some embodiments, the inner tubular extension and the connection region have an equal thickness when a lead or lead extension is not inserted into the lumen.

In at least some embodiments, the connection region is curved. In at least some embodiments, at least a portion of the inner tubular extension extends parallel to a longitudinal axis of the lumen. In at least some embodiments, at least a portion of the outer tubular extension extends parallel to a longitudinal axis of the lumen. In at least some embodiments, connector assembly further includes an end stop disposed at an end of the lumen.

Another embodiment is an electrical stimulating system including an electrical stimulation lead including a proximal end, a distal end, a plurality of terminals disposed along the proximal end, and a plurality of electrodes disposed along the distal end; and a control module coupleable to the electrical stimulation lead. The control module includes a housing, an electronic subassembly disposed in the housing; and any of the connector assemblies described above, where at least one of the connector contacts is electrically coupled to the electronic subassembly.

Yet another embodiment is a lead extension that includes any of the connector assemblies described above disposed on a first end of the lead extension; and terminals disposed along a second end of the lead extension.

A further embodiment is a lead assembly that includes a lead and the lead extension described above. Another embodiment is an electrical stimulation system that includes the lead assembly and a control module coupleable to the lead assembly. The control module includes a housing and an electronic subassembly disposed in the housing. In at least some embodiments, the control module includes any of the connector assemblies described above.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle body coupled to a control module via lead bodies, according to the invention;

FIG. 2 is a schematic view of another embodiment of an electrical stimulation system that includes a percutaneous lead body coupled to a control module via a lead body, according to the invention;

FIG. 3A is a schematic view of one embodiment of a plurality of connector assemblies disposed in the control module of FIG. 1, the connector assemblies configured to receive the proximal portions of the lead bodies of FIG. 1, according to the invention;

FIG. 3B is a schematic view of one embodiment of a connector assembly disposed in the control module of FIG. 2, the connector assembly configured to receive the proximal portion of one of the lead body of FIG. 2, according to the invention;

FIG. 3C is a schematic view of one embodiment of a proximal portion of the lead body of FIG. 2, a lead extension, and the control module of FIG. 2, the lead extension configured to couple the lead body to the control module, according to the invention;

FIG. 4 is a schematic, cross-sectional view of one embodiment of a connector assembly according to the invention;

FIG. 5A is a schematic, perspective view of a spacer, according to the invention;

FIG. 5B is a schematic, partially cut-away, perspective view of the spacer of FIG. 5A, according to the invention;

FIG. 5C is a schematic, cross-sectional view of the spacer of FIG. 5A, according to the invention;

FIG. 5D is a schematic, cross-sectional view of another embodiment of a spacer, according to the invention;

FIG. 5E is a schematic, partially cut-away, perspective view of yet another embodiment of a spacer, according to the invention;

FIGS. 6A-6F are a schematic, cross-sectional views of a portion of a spacer, connector contact, and a lead illustrating one embodiment of interaction between the spacer and the lead during insertion and retraction of the lead from a connector assembly, according to the invention; and

FIG. 7 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to connectors utilizing a novel spacer design, as well as methods of making and using the same.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties.

Examples of connectors, connector contacts and connector assemblies for electrical stimulation systems with leads are found in, for example, U.S. Pat. Nos. 8,849,396; 7,244,150; 8,600,507; 8,897,876; 8,682,439; U.S. Patent Applications Publication Nos. 2012/0053646; 2014/0148885; 2015/0209575; 2016/0059019; and U.S. Patent Provisional Patent Application Nos. 62/193,472; 62/216,594; 62/259,463; and 62/278,667, all of which are incorporated by reference in their entireties.

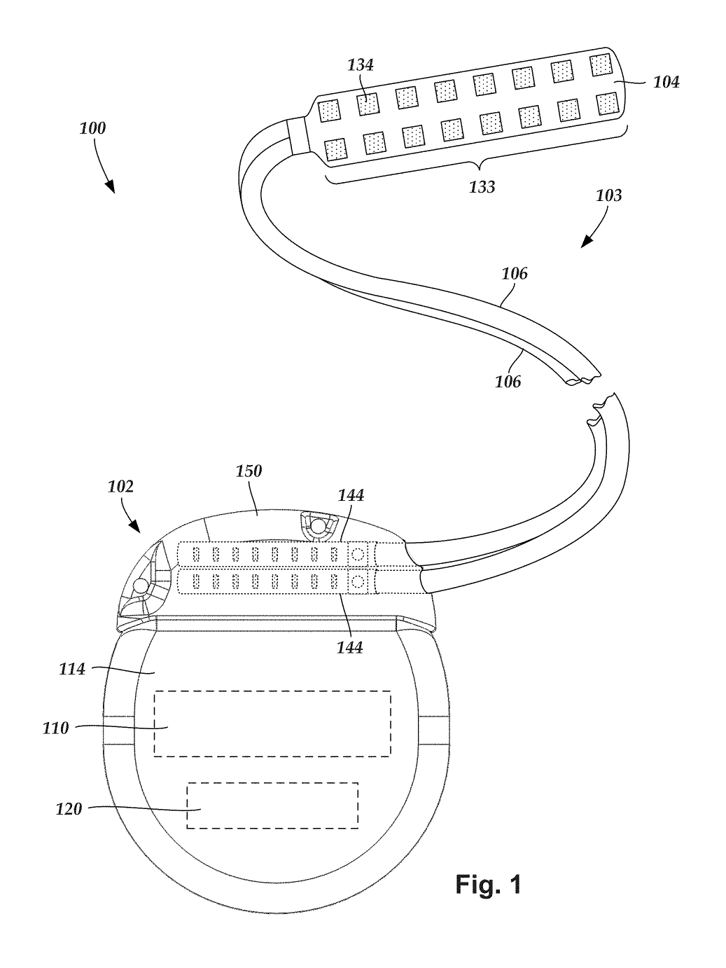

FIG. 1 illustrates schematically one embodiment of an electrical stimulation system 100. The electrical stimulation system includes a control module (e.g., a stimulator or pulse generator) 102 and a lead 103. The lead 103 including a paddle body 104 and one or more lead bodies 106 coupling the control module 102 to the paddle body 104. The paddle body 104 and the one or more lead bodies 106 form the lead 103. The paddle body 104 typically includes a plurality of electrodes 134 that form an array of electrodes 133. The control module 102 typically includes an electronic subassembly 110 and an optional power source 120 disposed in a sealed housing 114. In FIG. 1, two lead bodies 106 are shown coupled to the control module 102.

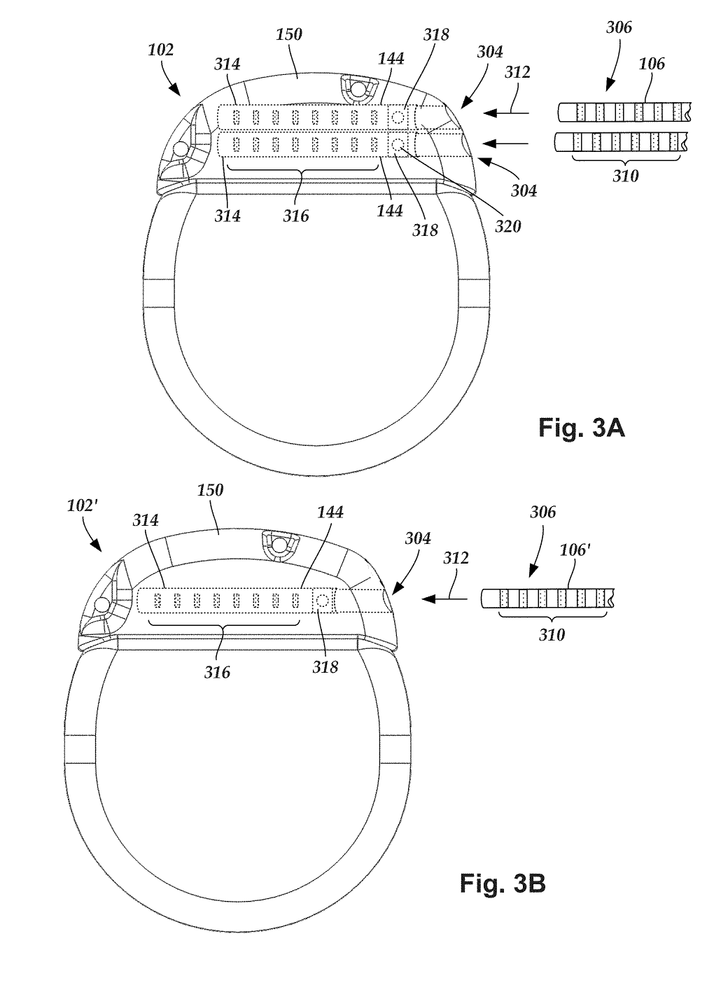

The control module 102 typically includes one or more connector assemblies 144 into which the proximal end of the one or more lead bodies 106 can be plugged to make an electrical connection via connector contacts (e.g., 316 in FIG. 3A) disposed in the connector assembly 144 and terminals (e.g., 310 in FIG. 3A) on each of the one or more lead bodies 106. The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 324 (see FIG. 3C) can be disposed between the one or more lead bodies 106 and the control module 102 to extend the distance between the one or more lead bodies 106 and the control module 102.

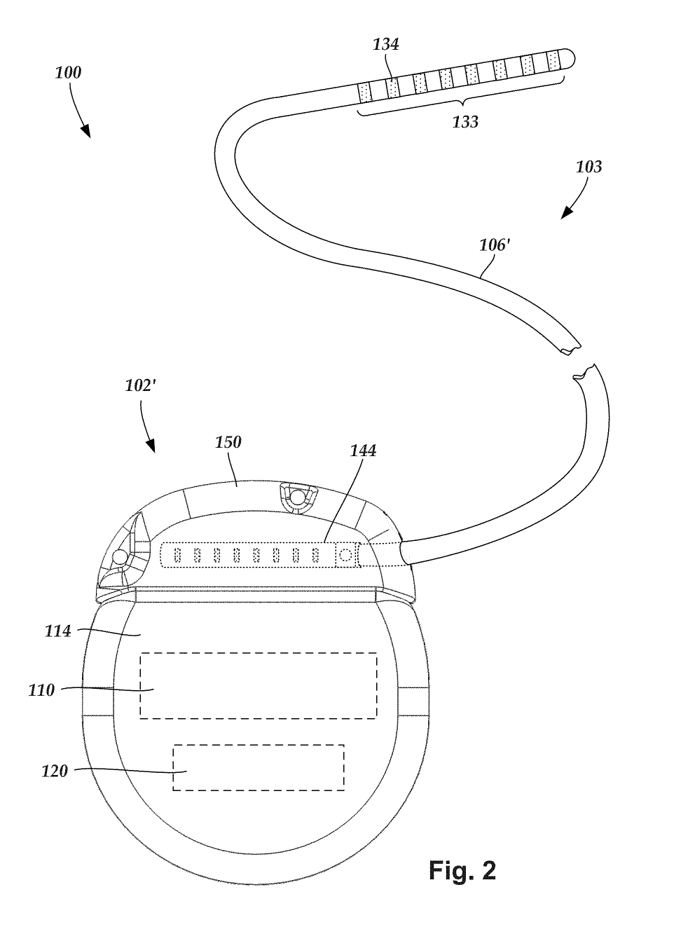

It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of a lead body 106′ forming a percutaneous lead 103, as illustrated in FIG. 2. The percutaneous lead may be isodiametric along the length of the lead body 106″. The lead body 106′ can be coupled with a control module 102′ with a single connector assembly 144.

The electrical stimulation system or components of the electrical stimulation system, including one or more of the lead bodies 106, the control module 102, and, in the case of a paddle lead, the paddle body 104, are typically implanted into the body of a patient. The electrical stimulation system can be used for a variety of applications including, but not limited to, spinal cord stimulation, brain stimulation, neural stimulation, muscle activation via stimulation of nerves innervating muscle, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, titanium, or rhenium.

The number of electrodes 134 in the array of electrodes 133 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead 103 to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A) are typically disposed at the proximal end of the one or more lead bodies 106 for connection to corresponding conductive contacts (e.g., 316 in FIG. 3A) in connector assemblies (e.g., 144 in FIG. 1) disposed on, for example, the control module 102 (or to other devices, such as conductive contacts on a lead extension, an operating room cable, a splitter, an adaptor, or the like).

Conductive wires (not shown) extend from the terminals (e.g., 310 in FIG. 3A) to the electrodes 134. Typically, one or more electrodes 134 are electrically coupled to a terminal (e.g., 310 in FIG. 3A). In some embodiments, each terminal (e.g., 310 in FIG. 3A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 3A is a schematic side view of one embodiment of a plurality of connector assemblies 144 disposed on the control module 102. In at least some embodiments, the control module 102 includes two connector assemblies 144. In at least some embodiments, the control module 102 includes four connector assemblies 144. In FIG. 3A, proximal ends 306 of the plurality of lead bodies 106 are shown configured for insertion to the control module 102. FIG. 3B is a schematic side view of one embodiment of a single connector assembly 144 disposed on the control module 102′. In FIG. 3B, the proximal end 306 of the single lead body 106′ is shown configured for insertion to the control module 102′.

In FIGS. 3A and 3B, the one or more connector assemblies 144 are disposed in the header 150. In at least some embodiments, the header 150 defines one or more ports 304 into which the proximal end(s) 306 of the one or more lead bodies 106/106′ with terminals 310 can be inserted, as shown by directional arrows 312, in order to gain access to the connector contacts disposed in the one or more connector assemblies 144.

The one or more connector assemblies 144 each include a connector housing 314 and a plurality of connector contacts 316 disposed therein. Typically, the connector housing 314 defines a port (not shown) that provides access to the plurality of connector contacts 316. In at least some embodiments, one or more of the connector assemblies 144 further includes a retaining element 318 configured to fasten the corresponding lead body 106/106′ to the connector assembly 144 when the lead body 106/106′ is inserted into the connector assembly 144 to prevent undesired detachment of the lead body 106/106′ from the connector assembly 144. For example, the retaining element 318 may include an aperture 320 through which a fastener (e.g., a set screw, pin, or the like) may be inserted and secured against an inserted lead body 106/106′.

When the one or more lead bodies 106/106′ are inserted into the one or more ports 304, the connector contacts 316 can be aligned with the terminals 310 disposed on the one or more lead bodies 106/106′ to electrically couple the control module 102 to the electrodes (134 of FIG. 1) disposed at a distal end of the one or more lead bodies 106. Examples of connector assemblies in control modules are found in, for example, U.S. Pat. Nos. 7,244,150 and 8,224,450, which are incorporated by reference.

In at least some embodiments, the electrical stimulation system includes one or more lead extensions. The one or more lead bodies 106/106′ can be coupled to one or more lead extensions which, in turn, are coupled to the control module 102/102′. In FIG. 3C, a lead extension connector assembly 322 is disposed on a lead extension 324. The lead extension connector assembly 322 is shown disposed at a distal end 326 of the lead extension 324. The lead extension connector assembly 322 includes a contact housing 328. The contact housing 328 defines at least one port 330 into which a proximal end 306 of the lead body 106′ with terminals 310 can be inserted, as shown by directional arrow 338. The lead extension connector assembly 322 also includes a plurality of connector contacts 340. When the lead body 106′ is inserted into the port 330, the connector contacts 340 disposed in the contact housing 328 can be aligned with the terminals 310 on the lead body 106 to electrically couple the lead extension 324 to the electrodes (134 of FIG. 1) disposed at a distal end (not shown) of the lead body 106′.

The proximal end of a lead extension can be similarly configured as a proximal end of a lead body. The lead extension 324 may include a plurality of conductive wires (not shown) that electrically couple the connector contacts 340 to terminal on a proximal end 348 of the lead extension 324. The conductive wires disposed in the lead extension 324 can be electrically coupled to a plurality of terminals (not shown) disposed on the proximal end 348 of the lead extension 324. In at least some embodiments, the proximal end 348 of the lead extension 324 is configured for insertion into a lead extension connector assembly disposed in another lead extension. In other embodiments (as shown in FIG. 3C), the proximal end 348 of the lead extension 324 is configured for insertion into the connector assembly 144 disposed on the control module 102′.

It will be understood that the control modules 102/102′ can receive either lead bodies 106/106′ or lead extensions 324. It will also be understood that the electrical stimulation system 100 can include a plurality of lead extensions 224. For example, each of the lead bodies 106 shown in FIGS. 1 and 3A can, alternatively, be coupled to a different lead extension 224 which, in turn, are each coupled to different ports of a two-port control module, such as the control module 102 of FIGS. 1 and 3A.

It will be understood that the connector assembly described below may be disposed in many different locations including, for example, on lead extensions (see e.g., 322 of FIG. 3C), lead adapters, lead splitters, the connector portion of control modules (see e.g., 144 of FIGS. 1-3B), or the like. In preferred embodiments, the connector assemblies are disposed on the distal ends of lead extensions.

A connector assembly in the control module or on a lead extension or other location can include an arrangement of connector contacts separated by spacers (which may also be referred to as seals). The spacers isolate or electrically insulate the connector contacts from each other and may also provide a seal with the lead to further isolate the connector contacts from each other. The spacers provide a sealing force or pressure on the lead body and array of terminals at an end of the lead or lead extension. Providing the seal increases the force for insertion of the lead or lead extension. The insertion force may result in difficulty inserting a lead, user dissatisfaction, or even lead damage due to high columnar loads. Moreover, as the number of electrodes on a lead increases, adding more connector contacts in a connector and terminals on the lead or lead extension will typically increase the insertion force. Therefore, it is desirable to develop spacer configurations with lower insertion force than conventional spacers.

A spacer can include an inner tubular extension and an outer tubular extension that are connected along or near one edge of each tubular extension while leaving another edge free to stretch or otherwise deform. In at least some embodiments, this spacer can provide a reliable seal against the lead or lead extension inserted into the connector and may also provide a seal against a housing of the connector.

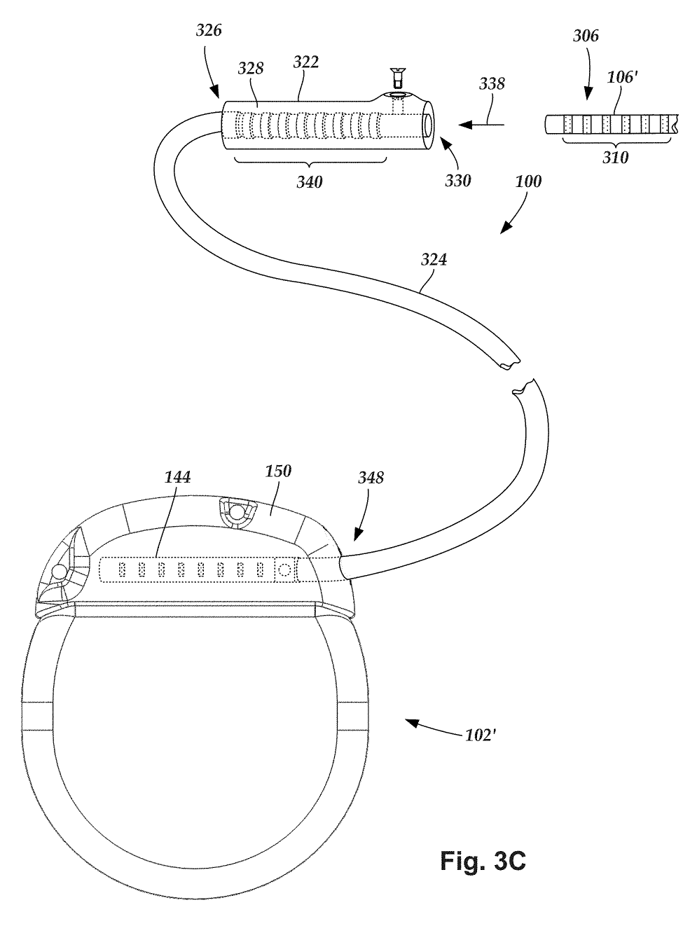

FIG. 4A shows a schematic, perspective view of a connector assembly 400 having a connector housing 402, connector contacts 404, spacers 406 that separate the connector contacts, an optional end stop 408, and an optional retention block 410. The connector housing 402 includes apertures 412 exposing the individual connector contacts 404 for attachment of a conductor (e.g., a wire—not shown) to the connector contact. In at least some embodiments, the apertures 412 in a finished connector assembly 400 are filled after or during attachment of the conductors to the connector contacts 404.

The connector housing 402 defines a port 414 that provides access to a connector lumen 416 and the connector contacts 404. The connector housing 402 can be made of any suitable material or materials. In at least some embodiments, the connector assembly 400 further includes a retention block 410 to fasten the corresponding lead body (or a retention ring on the lead body) of the lead or lead extension to the connector assembly 400 when the lead body is inserted into the connector assembly and prevent undesired detachment of the lead body from the connector assembly or misalignment of the terminals on the lead body with the connector contacts. For example, the retaining element 318 may include an aperture 418 through which a fastener (e.g., a set screw, pin, or the like) may be inserted and secured against an inserted lead body. Other types of retention blocks or retention assemblies can be used including, but not limited to, those described in U.S. Pat. No. 9,440,066; U.S. patent application Ser. Nos. 15/627,016 and 15/641,688; and U.S. Provisional Patent Application Ser. No. 62/464,710, all of which are incorporated herein by reference.

The connector contacts 404 may take the form of conductive spring contacts or any other suitable contact arrangement. Examples of connector contacts include, but are not limited to, canted coil contacts available from Bal Seal Engineering, Inc. (Foothill Ranch, Calif.) and contacts described in U.S. Pat. Nos. 7,803,021; 8,682,439; 8,897,876; 9,409,032; 9,604,068; 9,656,093; and 9,770,598; U.S. Patent Application Publications Nos. 2011/0022100; 2016/0228692; and 2016/0296745; U.S. patent application Ser. Nos. 15/627,016 and 15/656,612; and U.S. Provisional Patent Application Ser. No. 62/483,141, all of which are incorporated herein by reference.

The connector assembly 400 may include an end stop 408 which, at least in part, modulates insertion of the lead or lead extension into the port 414. The end stop 408 can be disposed in the lumen 416 of the connector assembly 400. The end stop 408 can provide one or more surfaces upon which the inserted lead or lead extension contacts, when the lead or lead extension is fully inserted into the port 414. In some cases, the end stop 408 can provide the proximal-most point of insertion for the lead or lead extension within the connector assembly 400.

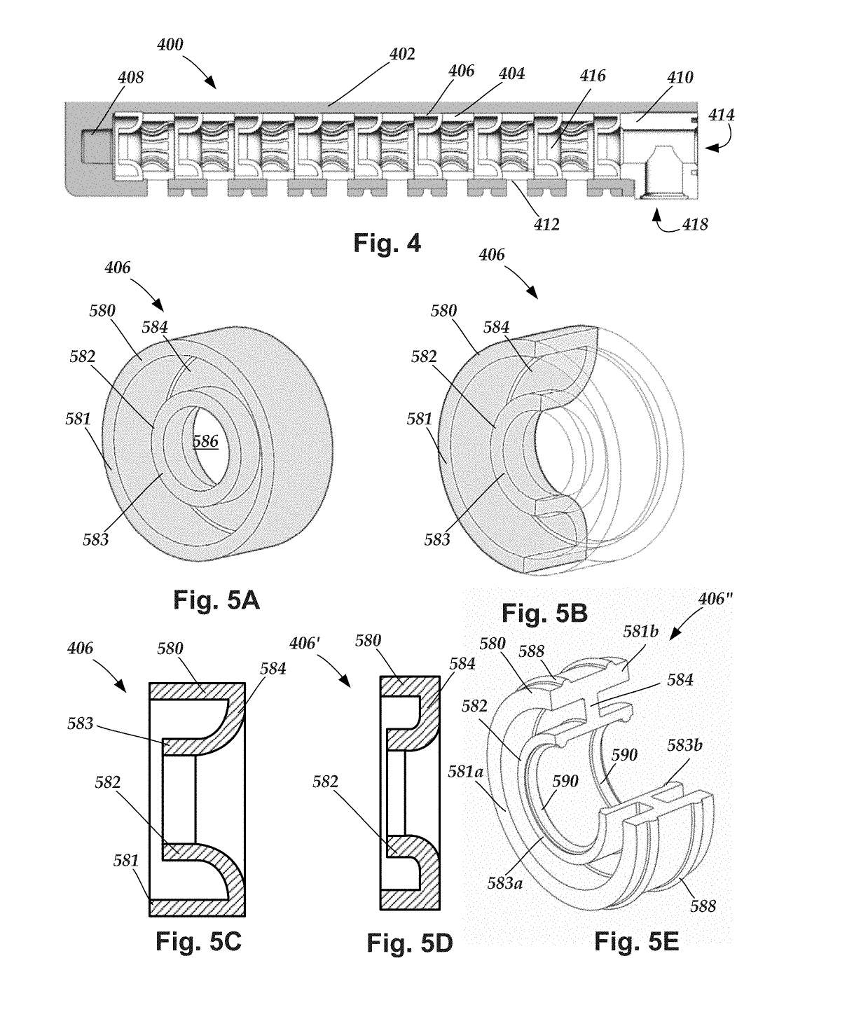

FIGS. 5A-5C illustrate schematic views of one embodiment of a spacer 406. The spacer 406 can be described as having an outer tubular extension 580, an inner tubular extension 582, and a connection region 584 connecting the outer tubular extension 580 to the inner tubular extension 582. An end 581, 583 of each of the outer and inner tubular extensions 580, 582 is free. As described below, the free end 583 of the inner tubular extension 582 can be stretched, displaced, or otherwise deformed as the lead is inserted into the connector assembly 400. In at least some embodiments, the connection region 584 can be viewed as coupling a second end of the inner tubular extension 582 to a second end of the outer tubular extension 580.

In the illustrated embodiments, the inner and outer tubular extensions 582, 580 have a circular or ring-like cross-sectional shape, but other forms can also be used including shapes with oval, rectangular, triangular, octagonal, or hexagonal cross-sections or the like. In the illustrated embodiments, the inner and outer tubular extensions 582, 580 have at least a portion that extends parallel to a longitudinal axis of the lumen 416 of the connector assembly (FIG. 4), but it will be recognized that other embodiments of the spacer may have an inner tubular extension or an outer tubular extension that is sloped relative to the longitudinal axis of the lumen of the connector assembly or has any other suitable shape. In the illustrated embodiment, the connection region 504 is curved, but in other embodiments, a portion (or all of) the connection region 504 may be straight or have any other suitable shape.

The spacer 406 can be made of any suitable flexible, non-conductive material including, but not limited to, silicone, polyurethane, or the like. The material of the spacer 406 is preferably stretchable. In at least some embodiments, the spacer 406 is formed by molding.

The inner tubular extension 582 defines a lumen 586 through which a portion of the lead or lead extension extends when the inserted into the connector assembly 400. The inner diameter of the inner tubular extension 582 is preferably equal to, or slightly smaller (for example, no more than 15%, 10%, or 5% smaller) than, the diameter of the lead or lead extension to be inserted into the connector assembly 400. In at least some embodiments, the inner tubular extension 582 makes a seal (preferably, a hermetic seal) with the portion of the lead or lead extension inserted into the connector assembly 400. In at least some embodiments, a ratio of sealing force to insertion force is at least 1.5, 1.6, 1.7, or 1.8. This ratio can be determined using a finite element analysis.

Although not wishing to be bound to any particular theory, the following is a description of one method of analyzing the sealing force and the insertion force. In at least some embodiments, the insertion force can be considered the combination of two forces: displacement and friction. Displacement is the force generated by moving the inner tubular extension. As one example of a determination of the displacement force, when the inner tubular extension is bent, the displacement force is proportional to the product of the displacement, the elastic modulus, and the second polar moment of inertia divided by the length of the bending element (e.g., F_{dis}=d*E*I/L). In at least some embodiments, when the flange is stretched, the displacement can be modeled using Hook's law with the force equal to the product of the stretching distance and a material property, k, (e.g., F=kx). The “stretch” in this case is the portion connecting to the inner and outer tubular extensions, not the circumferential stretch of the inner tubular extension. In at least some embodiments, friction is equal to the product of the normal force and a friction coefficient, μ (e.g., F=μN). In at least some embodiments, the normal force is the sealing force and may be, for example, derived from the radial component of a force related to the circumferential stretching of the inner tubular extension (which may also be calculated using Hook's law). The friction coefficient depends on the materials of the spacer and the lead, in combination, and other factors such surface texture and possibly lubricity. Calculation of these forces in 360 degrees with multiple interactions between these forces can be complicated and challenging, but may be modeled.

In at least some embodiments, the outer diameter of the outer tubular extension 584 is equal to or slightly larger (for example, no more than 15%, 10%, or 5% larger) than, the inner diameter of the connector housing 402 of the connector assembly 400. In at least some embodiments, the outer tubular extension 584 makes a seal (preferably, a hermetic seal) with the connector housing 402 of the connector assembly 400. In at least some embodiments, the outer tubular extension 584 makes a seal (preferably, a hermetic seal) with the adjacent connector contacts 404 (or with an adjacent connector contact 404 and the end stop 408 or retention block 410) of the connector assembly 400.

In at least some embodiments, the thicknesses of the inner tubular extension 582 and the outer tubular extension 580 are equal or differ by no more than 5%, 10%, or 20%. In at least some embodiments, the thicknesses of the inner tubular extension 582 and the connection region 584 are equal or differ by no more than 5%, 10%, or 20%. In the illustrated embodiments, the outer tubular extension 580 extends further in the axial direction than the inner tubular extension 582 when there is no lead or lead extension in the connector assembly, but it will be recognized that the outer and inner tubular extensions may have the same axial extent, or the inner tubular extension may extend further axially than the outer tubular extension in other embodiments.

FIG. 5C is a cross-sectional view of the spacer 406. FIG. 5D is a cross-sectional view of another embodiment of a spacer 406′ that has a smaller axial length than the spacer 406.

FIG. 5E illustrates yet another embodiment of a spacer 406″ with an outer tubular extension 580, an inner tubular extension 582, and a connection region 584 connecting the outer tubular extension 580 to the inner tubular extension 582. In this embodiment, the connection region 584 couples an intermediation region of the outer tubular extension 580 to an intermediate region of the inner tubular extension 582. The outer tubular extension 580 has two free ends 581a, 581b and the inner tubular extension 582 has two free ends 583a, 583b providing a cross-sectional shape similar to the letter “H”. The connection region 584 may couple the outer tubular extension 580 to the inner tubular extension 582 at the center of the respective tubular extension or anywhere along the respective longitudinal lengths of the tubular extensions and may be centered with respect to both the inner and outer tubular extensions or non-centered with respect to one or both of the inner and outer tubular extensions. Moreover, in other embodiments, the connection region 584 may be coupled to different portions of the outer and inner tubular extension, such as, for example, coupled to a center intermediate region of one of the tubular extensions and coupled to an intermediate region that is not centered for the other one of the tubular extensions. The connection region 584 in FIG. 5E is illustrated as extending in a straight radial direction, but, in other embodiments, the connection region may be slanted or curved (see, for example, FIG. 5B) with respect to the radial direction.

In yet other embodiments, the spacer may have a cross-sectional shape similar to the letter “h” with either 1) the outer tubular extension having a single free end (as illustrated in FIG. 5A) and the inner tubular extension having two free ends (as illustrated in FIG. 5E) or 2) the outer tubular extension having two free ends (as illustrated in FIG. 5E) and the inner tubular extension having a single free end (as illustrated in FIG. 5A).

Any of the embodiments described herein can include one or more radial protuberances 588 extending around a portion of (or the entire) perimeter of the outer surface of the outer tubular extension 580, as illustrated in FIG. 5E. The illustrated embodiment in FIG. 5E has two protuberances 588 that extend around the entire perimeter of the outer surface of the outer tubular extension, but it will be recognized that any other number of protuberances (e.g., one, three, four, or more) can be used and that the protuberances may only extend around a portion of the perimeter or may be separated into multiple segments (for example, two, three, four, or more segments) that each extend around only a portion of the perimeter. The protuberances 588 may facilitate forming a seal with the connector housing 402.

Any of the embodiments described herein can include one or more radial protuberances 590 extending around a portion of (or the entire) perimeter of the inner surface of the inner tubular extension 590, as illustrated in FIG. 5E. The illustrated embodiment in FIG. 5E has two protuberances 590 that extend around the entire perimeter of the inner surface of the outer tubular extension, but it will be recognized that any other number of protuberances (e.g., one, three, four, or more) can be used and that the protuberances may only extend around a portion of the perimeter or may be separated into multiple segments (for example, two, three, four, or more segments) that each extend around only a portion of the perimeter. The protuberances 590 may facilitate forming a seal with the lead or lead extension inserted into the connector assembly 400.

In any of the embodiments, the walls of the inner tubular extension or outer tubular extension may be tapered towards the ends. In any of the embodiments, the inner tubular extension may be shorter in width to be stiffer and further resist buckling. Optionally, radial walls or spokes may be added between the inner and outer tubular extensions to further resist buckling.

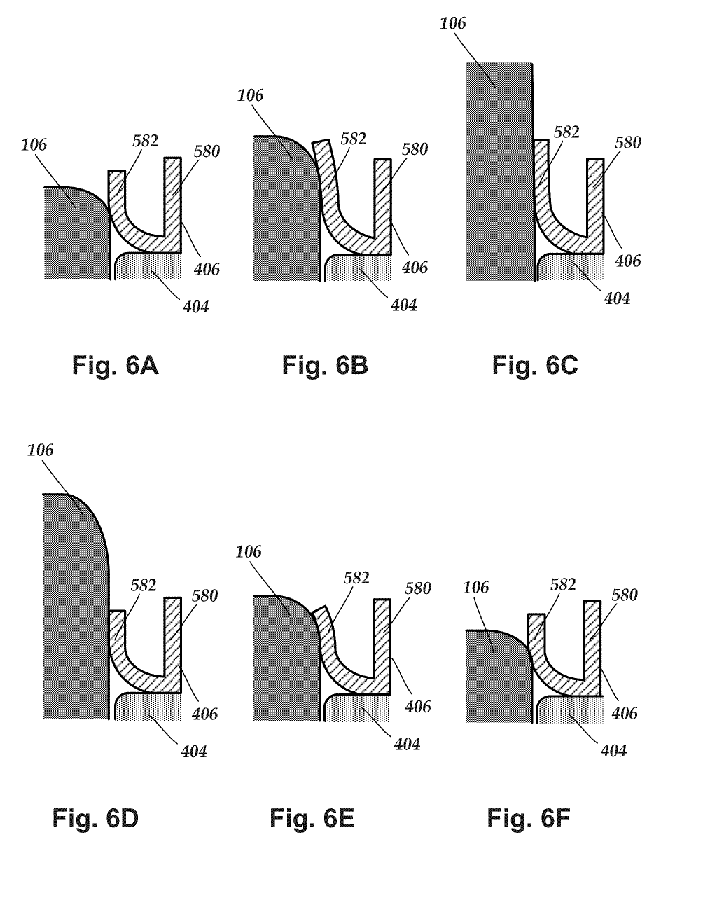

FIGS. 6A-6F are cross-sectional views of a portion of a spacer 406, lead 106, and contact 404 during the insertion (FIGS. 6A-6C) and retraction (FIGS. 6D-6F) of the lead 106 into/out of a connector assembly. These FIGS. 6A-6F illustrate one embodiment of the alterations to the spacer 406 during the insertion/retraction processes. It will be recognized that other spacers of the invention made of different materials or having different forms may move, stretch, or otherwise deform differently.

In FIG. 6A, the end of the lead 106 makes contact with the spacer 406. As the lead 106 is pushed past the spacer 406, the inner tubular extension 582 of the spacer may stretch or deform, as illustrated in FIG. 6B. As the lead 106 is fully inserted into the connector assembly, the inner tubular extension 582 may be stretched substantially from its original shape, as illustrated in FIG. 5C. In some embodiments, an adjacent connector contact 404 (FIG. 4) or end stop 408 (FIG. 4) may prevent or reduce further stretching of the inner tubular extension 582. Preferably, the inner tubular extension 582 makes a seal (more preferably, a hermetic seal) with the lead 106.

As the lead is retracted, the inner tubular extension 582 moves back toward the connector contact 404, as illustrated in FIGS. 6D and 6E, and may deform. In at least some embodiments, the connector contact 404 may prevent or hinder the inner tubular extension 582 from rolling backward and inverting. As the end of the lead 106 moves past spacer 406, the inner tubular extension 582 may return to its original shape, as illustrated in FIG. 6F, (although, in some embodiments, there may be residual deformation or stretching or other inelastic changes to the spacer shape).

In FIGS. 4, 5A to 5D, and 6A to 6F, the inner tubular extension 582 extends away from the connection region 584 toward the proximal end of the connector assembly 400 where the optional end stop 408 may reside, as illustrated in FIG. 4. It will be recognized, however, that in other embodiments, the arrangement of the spacers 406 can be reversed so that the inner tubular extension 582 extends away from the connection region 584 toward the distal end of the connector assembly 400 where the port 414 resides. Such an arrangement may lower the insertion force of the lead (and increasing the seal-to-insertion force ratio) as compared to the arrangement of the spacers illustrated in FIG. 4.

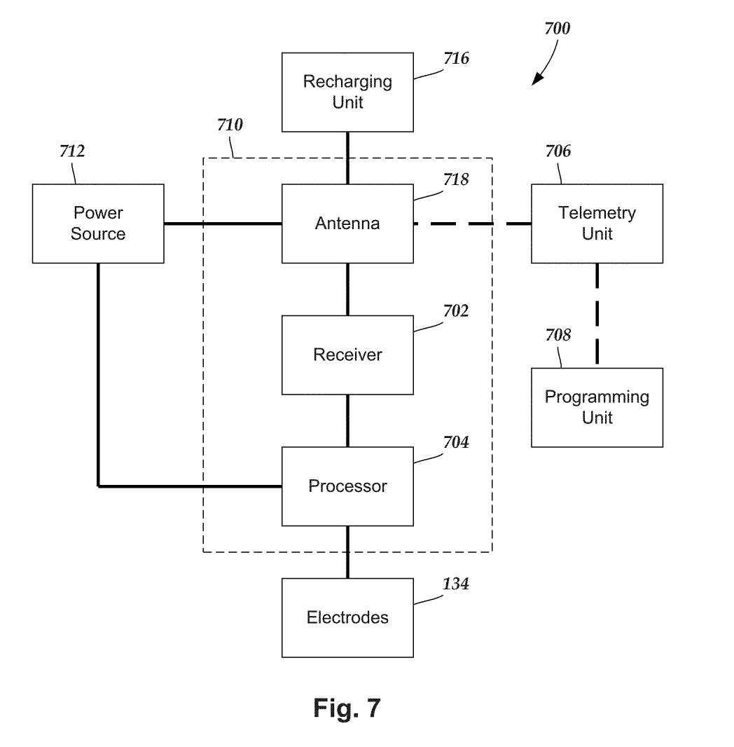

FIG. 7 is a schematic overview of one embodiment of components of an electrical stimulation system 700 including an electronic subassembly 710 disposed within a control module. It will be understood that the electrical stimulation system can include more, fewer, or different components and can have a variety of different configurations including those configurations disclosed in the stimulator references cited herein.

Some of the components (for example, a power source 712, an antenna 718, a receiver 702, and a processor 704) of the electrical stimulation system can be positioned on one or more circuit boards or similar carriers within a sealed housing of an implantable pulse generator, if desired. Any power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 704 is generally included to control the timing and electrical characteristics of the electrical stimulation system. For example, the processor 704 can, if desired, control one or more of the timing, frequency, strength, duration, and waveform of the pulses. In addition, the processor 704 can select which electrodes can be used to provide stimulation, if desired. In some embodiments, the processor 704 selects which electrode(s) are cathodes and which electrode(s) are anodes. In some embodiments, the processor 704 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 708 that, for example, allows modification of pulse characteristics. In the illustrated embodiment, the processor 704 is coupled to a receiver 702 which, in turn, is coupled to the optional antenna 718. This allows the processor 704 to receive instructions from an external source to, for example, direct the pulse characteristics and the selection of electrodes, if desired.

In one embodiment, the antenna 718 is capable of receiving signals (e.g., RF signals) from an external telemetry unit 706 which is programmed by the programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the electrical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and the receiver 702 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 700 to cease operation, to start operation, to start charging the battery, or to stop charging the battery. In other embodiments, the stimulation system does not include the antenna 718 or receiver 702 and the processor 704 operates as programmed.

Optionally, the electrical stimulation system 700 may include a transmitter (not shown) coupled to the processor 704 and the antenna 718 for transmitting signals back to the telemetry unit 706 or another unit capable of receiving the signals. For example, the electrical stimulation system 700 may transmit signals indicating whether the electrical stimulation system 700 is operating properly or not or indicating when the battery needs to be charged or the level of charge remaining in the battery. The processor 704 may also be capable of transmitting information about the pulse characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A connector assembly, comprising:
an elongated connector housing having a first end, a second end, and a length, the connector housing defining a port at the second end of the connector housing, the port configured for receiving a proximal end of a lead or lead extension;
a lumen that extends from the port along at least a portion of the length of the connector housing;
a plurality of connector contacts axially spaced-apart and disposed along the lumen such that the connector contacts are each exposed to the lumen, the connector contacts configured for coupling to a proximal end of a lead or lead extension when the proximal end of the lead or lead extension is inserted into the lumen; and
a plurality of non-conductive spacers disposed between adjacent connector contacts, each of the spacers comprising an outer tubular extension, an inner tubular extension, and a connection region coupling the outer tubular extension to the inner tubular extension, wherein the inner tubular extension has a free end and the inner tubular extension is configured and arranged to form a seal against the proximal end of the lead or lead extension when inserted into the lumen.

2. The connector assembly of claim 1, wherein the outer tubular extension is configured and arranged to form a seal against the connector housing.

3. The connector assembly of claim 1, wherein the inner tubular extension forms a ring.

4. The connector assembly of claim 1, wherein the outer tubular extension forms a ring.

5. The connector assembly of claim 1, wherein the inner tubular extension and the outer tubular extension have an equal thickness when a lead or lead extension is not inserted into the lumen.

6. The connector assembly of claim 1, wherein the outer tubular extension has a free end.

7. The connector assembly of claim 1, wherein the inner tubular extension or the outer tubular extension or both the inner and outer tubular extensions have two free ends and the connection region couples an intermediate portion of the outer tubular extension to an intermediate portion of the inner tubular extension.

8. The connector assembly of claim 1, wherein the inner tubular extension is configured and arranged to stretch when the proximal end of the lead or lead extension is inserted into the lumen.

9. The connector assembly of claim 1, wherein the connector assembly is configured and arranged so that a one of the connector contacts acts as a stop to stretching of the inner tubular extension of a one of the spacers as the proximal end of the lead or lead extension is inserted into the lumen.

10. The connector assembly of claim 1, wherein the connector assembly is configured and arranged so that a one of the connector contacts acts as a stop to retraction of the inner tubular extension of a one of the spacers as the proximal end of the lead or lead extension is removed into the lumen.

11. The connector assembly of claim 1, wherein the outer tubular extension extends further in an axial direction than the inner tubular extension.

12. The connector assembly of claim 1, wherein the inner tubular extension and the connection region have an equal thickness when a lead or lead extension is not inserted into the lumen.

13. The connector assembly of claim 1, wherein the connection region is curved.

14. The connector assembly of claim 1, wherein at least a portion of the inner tubular extension extends parallel to a longitudinal axis of the lumen.

15. The connector assembly of claim 1, wherein at least a portion of the outer tubular extension extends parallel to a longitudinal axis of the lumen.

16. An electrical stimulating system comprising:
an electrical stimulation lead comprising a proximal end, a distal end, a plurality of terminals disposed along the proximal end, and a plurality of electrodes disposed along the distal end; and
a control module coupleable to the electrical stimulation lead, the control module comprising
a housing,
an electronic subassembly disposed in the housing; and
the connector assembly of claim 1, wherein at least one of the connector contacts is electrically coupled to the electronic subassembly.

17. A lead extension, comprising
the connector assembly of claim 1 disposed on a first end of the lead extension; and
a plurality of terminals disposed along a second end of the lead extension.

18. A lead assembly, comprising:
a lead; and
the lead extension of claim 17.

19. An electrical stimulation system, comprising:
the lead assembly of claim 18; and
a control module coupleable to the lead assembly, the control module comprising
a housing, and
an electronic subassembly disposed in the housing.

20. The electrical stimulation system of claim 19, wherein the control module comprises a connector assembly comprising:
an elongated connector housing having a first end, a second end, and a length, the connector housing defining a port at the second end of the connector housing, the port configured for receiving a proximal end of a lead or lead extension;
a lumen that extends from the port along at least a portion of the length of the connector housing;
a plurality of connector contacts axially spaced-apart and disposed along the lumen such that the connector contacts are each exposed to the lumen, the connector contacts configured for coupling to a proximal end of a lead or lead extension when the proximal end of the lead or lead extension is inserted into the lumen; and
a plurality of non-conductive spacers disposed between adjacent connector contacts, each of the spacers comprising an outer tubular extension, an inner tubular extension, and a connection region coupling the outer tubular extension to the inner tubular extension, wherein the inner tubular extension has a free end and the inner tubular extension is configured and arranged to form a seal against the proximal end of the lead or lead extension when inserted into the lumen.
